(19) United States
(12) Patent Application Publication
(10) Pub. No.: US 2005/0081860 A1
(43) Pub. Date: Apr. 21, 2005

(54) Tracheostomy care container system

(76) Inventor: Rosa Gonzales, Phoenix, AZ (US)

Correspondence Address: STONEMAN LAW OFFICES, LTD, 3113 NORTH 3RD STREET, PHOENIX, AZ, 85012, US

(21) Appl. No.: 10/688,491

(22) Filed: Oct. 16, 2003

(51) Int. Cl. A61M 15/00; A61M 16/00

(52) U.S. Cl. 128/207.14

(57) ABSTRACT

A tracheostomy care container system for assisting in organizing, managing, and transporting trach care materials. It can be used to store a used suction catheter in a clean environment so that the catheter can be used again, and also allows a user to easily distinguish between cleaning solution and water used during trach care.

### BACKGROUND

This invention relates to providing a system to assist users in more conveniently and efficiently providing tracheostomy (also called tracheotomy or trach) care. People with a tracheostomy need to have the skin around the stoma cleaned to reduce the chance for infection and to keep the skin under the tracheostomy tube and ties healthy. Often, people with a tracheostomy need to use a tracheostomy suction catheter multiple times a day to have mucus suctioned from their lungs and trachea. The term “catheter” refers to such a tracheostomy suction catheter.

Although it is typically safer to open a sterile package and use a new catheter each time suctioning is performed, catheters are relatively expensive. It would be much more cost effective to clean and store catheters so that they can be used several times. However, in the past there has not been a convenient and efficient way to store used catheters in a clean environment, especially adjacent the other materials and instruments used in performing trach care. For example, placing a catheter on a table (or other unclean surface) can contaminate the catheter, thereby placing the trach patient at risk for infection if the catheter is used again.

In addition, typically, trach care involves using two liquids: first, a cleaning solution (typically hydrogen peroxide diluted with water); and second, pure water. Typically, in preparation for performing trach care, the liquids are poured into separate disposable cups: one for cleaning solution; and the other for pure water. In the past, mistakes in trach care have been made due to confusion over mistaking the cleaning solution for the water, and vice-versa, since both liquids appear similar. Further, in the past it has been easy to accidentally knock over one of the disposable cups (spilling the contents) in the process of performing trach care.

### OBJECTS AND FEATURES OF THE INVENTION

A primary object and feature of the present invention is to provide a system to assist in organizing and managing trach care materials.

It is a further object and feature of the present invention to provide such a system which can be used to conveniently transport trach care materials. It is a further object and feature of the present invention to provide such a system which can be used to conveniently hold trach care materials in a location near to where they will be used.

It is a further object and feature of the present invention to provide such a system which can be used to store a used suction catheter in a clean environment so that the catheter can be used again.

It is a further object and feature of the present invention to provide such a system which allows a user to easily distinguish between cleaning solution and water. It is a further object and feature of the present invention to provide such a system which holds the cleaning solution and water so that they are not easily spilled.

A further primary object and feature of the present invention is to provide such a system that is efficient, inexpensive, and handy. Other objects and features of this invention will become apparent with reference to the following descriptions.

### SUMMARY OF THE INVENTION

In accordance with a preferred embodiment hereof, this invention provides a tracheostomy care container system comprising, in combination: at least one suction catheter kit container adapted to contain a plurality of suction catheter kits; and at least one used suction catheter container adapted to contain a used suction catheter within a substantially clean environment so that such used suction catheter can be used again. Moreover, it provides such a tracheostomy care container system further comprising at least one suction catheter holder to hold such used suction catheter within such at least one used suction catheter container. Additionally, it provides such a tracheostomy care container system wherein such at least one suction catheter holder comprises hook-and-loop fastening material. Also, it provides such a tracheostomy care container system further comprising: at least one tracheostomy care item container adapted to contain at least one tracheostomy care item; wherein such at least one tracheostomy care item is selected from the group consisting of gauze, a cotton swab, a hemostat, a saline vial, a tracheostomy tube holder, a tracheostomy tube, medical tape, lubricant jelly.

In addition, it provides such a tracheostomy care container system further comprising: at least one gauze pad, at least one cotton swab, at least one tracheostomy tube holder, at least one tracheostomy tube, a reservoir for water, and a reservoir for cleaning solution. And, it provides such a tracheostomy care container system wherein such at least one used suction catheter container is adapted to nest within such at least one suction catheter kit container. Further, it provides such a tracheostomy care container system further comprising at least one carrying assistor selected from the group consisting of a shoulder strap and a handle. Even further, it provides such a tracheostomy care container system further comprising at least one attacher structured and arranged so that the tracheostomy care container system can be attached to hang from furniture. Moreover, it provides such a tracheostomy care container system further comprising: at least one water holder adapted to hold at least one water container; and at least one cleaning solution holder adapted to hold at least one cleaning solution container. Additionally, it provides such a tracheostomy care container system further comprising at least one tracheostomy care item container adapted to contain at least one tracheostomy care item, wherein such at least one tracheostomy care item is selected from the group consisting of gauze, a cotton swab, a hemostat, a saline vial, a tracheostomy tube holder, an tracheostomy tube, medical tape, and lubricant jelly.

Also, it provides such a tracheostomy care container system wherein such at least one suction catheter kit container comprises at least one dispensing slot adapted to dispense suction catheter kits. In addition, it provides such a tracheostomy care container system wherein such suction catheter kit container comprises: a suction catheter kit container length, wherein such suction catheter kit container length is about 10 inches; and a suction catheter kit container width, wherein such suction catheter kit container width is about 5-1/2 inches. And, it provides such a tracheostomy care container system further comprising: at least one cotton swab container adapted to contain at least one cotton swab, wherein such at least one cotton swab container comprises a reclosable lid; at least one gauze pad container adapted to contain at least one gauze pad; and at least one hydrogen peroxide container adapted to contain at least one bottle of hydrogen peroxide. Further, it provides such a tracheostomy care container system further comprising: at least one hemostat container adapted to contain at least one hemostat; at least one tracheostomy tube holder container adapted to contain at least one tracheostomy tube holder; and at least one tracheostomy tube container adapted to contain at least one tracheostomy tube.

In accordance with another preferred embodiment hereof, this invention provides a tracheostomy care container system comprising, in combination: at least one suction catheter kit container adapted to contain a plurality of suction catheter kits; wherein such suction catheter kit container comprises a suction catheter kit container length, wherein such suction catheter kit container length is about as long as a standard suction catheter kit, a suction catheter kit container width, wherein such suction catheter kit container width is about as wide as a standard suction catheter kit; and at least one carrying assistor selected from the group consisting of, a shoulder strap, and a handle. Even further, it provides such a tracheostomy care container system wherein such at least one carrying assistor is structured and arranged to be detachable. Moreover, it provides such a tracheostomy care container system wherein such suction catheter kit container length is about 10 inches, and wherein such suction catheter kit container width is about 5-1/2 inches.

In accordance with another preferred embodiment hereof, this invention provides a tracheostomy care container system, which can be attached to furniture, comprising, in combination: at least one suction catheter kit container adapted to contain a plurality of suction catheter kits; and at least one attacher structured and arranged so that the tracheostomy care container system can be attached to hang from the furniture. Additionally, it provides such a tracheostomy care container system wherein such at least one attacher comprises at least one lip. Also, it provides such a tracheostomy care container system wherein such at least one lip is adapted for attachment to the upper rail of a crib.

In accordance with another preferred embodiment hereof, this invention provides a tracheostomy care container system comprising, in combination: at least one suction catheter kit container adapted to contain a plurality of suction catheter kits; and at least one used suction catheter container adapted to contain a used suction catheter within a substantially sterile environment so that such used suction catheter can be used again; wherein such at least one suction catheter kit container comprises at least one dispensing slot adapted to dispense suction catheter kits. In addition, it provides such a tracheostomy care container system wherein such at least one used suction catheter container is adapted to nest within such at least one suction catheter kit container.

In accordance with another preferred embodiment hereof, this invention provides a tracheostomy care container system comprising, in combination: at least one suction catheter kit container adapted to contain at least one suction catheter kit; at least one water holder adapted to hold at least one water container; and at least one cleaning solution holder adapted to hold at least one cleaning solution container. And, it provides such a tracheostomy care container system wherein such at least one water holder comprises at least one water container; and wherein such at least one cleaning solution holder comprises at least one cleaning solution container. Further, it provides such a tracheostomy care container system wherein such at least one water container comprises at least one cup; and wherein such at least one cleaning solution container comprises at least one cup. Even further, it provides such a tracheostomy care container system wherein such at least one water holder is adapted to removably hold at least one disposable cup; and wherein such at least one cleaning solution holder is adapted to removably hold at least one disposable cup. Even further, it provides such a tracheostomy care container system wherein such at least one water holder comprises water identification indicia; and wherein such at least one cleaning solution holder comprises cleaning solution identification indicia. Even further, it provides such a tracheostomy care container system wherein such water identification indicia comprise at least one different color than such cleaning solution identification indicia. Even further, it provides such a tracheostomy care container system wherein such water identification indicia comprises primarily the color blue. Even further, it provides such a tracheostomy care container system wherein such cleaning solution identification indicia comprises primarily the color red. Even further, it provides such a tracheostomy care container system wherein such water identification indicia comprise at least one word.

### BRIEF DESCRIPTION OF THE DRAWINGS

FIG. 1 shows a perspective view of a tracheostomy care container system, along with some trach care items, according to a preferred embodiment of the present invention.

FIG. 2 shows a top view of the tracheostomy care container system of FIG. 1.

FIG. 3 shows a perspective view of the tracheostomy care container system of FIG. 1.

FIG. 4 shows a perspective view of the tracheostomy care container system (without trach care items) of FIG. 1.

FIG. 5 shows a collection of trach care items which comprise a tracheostomy care container system according to the illustrated preferred embodiment of the present invention.

FIG. 6 shows a partial cutaway of the tracheostomy care container system of FIG. 1, showing a dispensing slot for clean sealed containers holding suction catheters.

### DETAILED DESCRIPTION OF THE BEST MODE AND PREFERRED EMBODIMENTS OF THE INVENTION

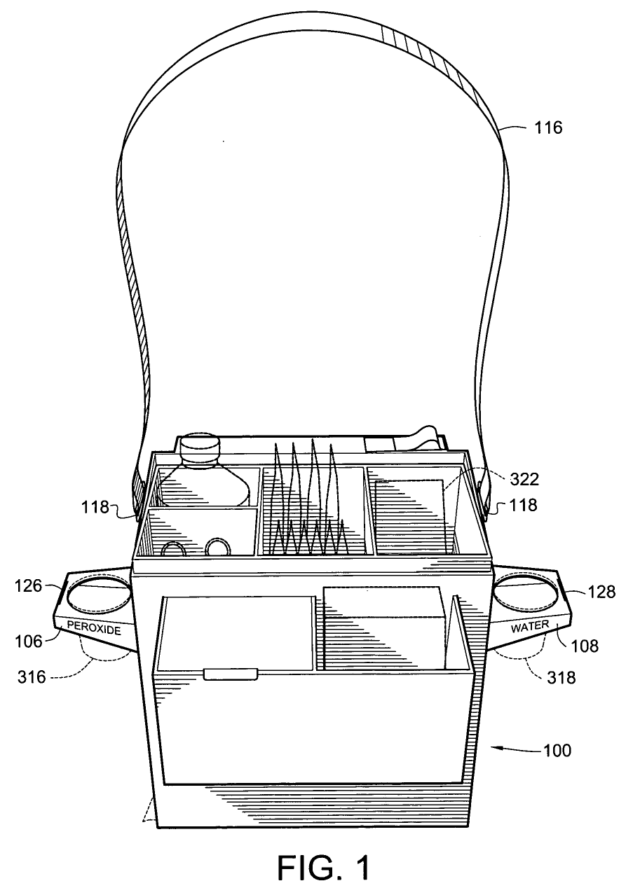

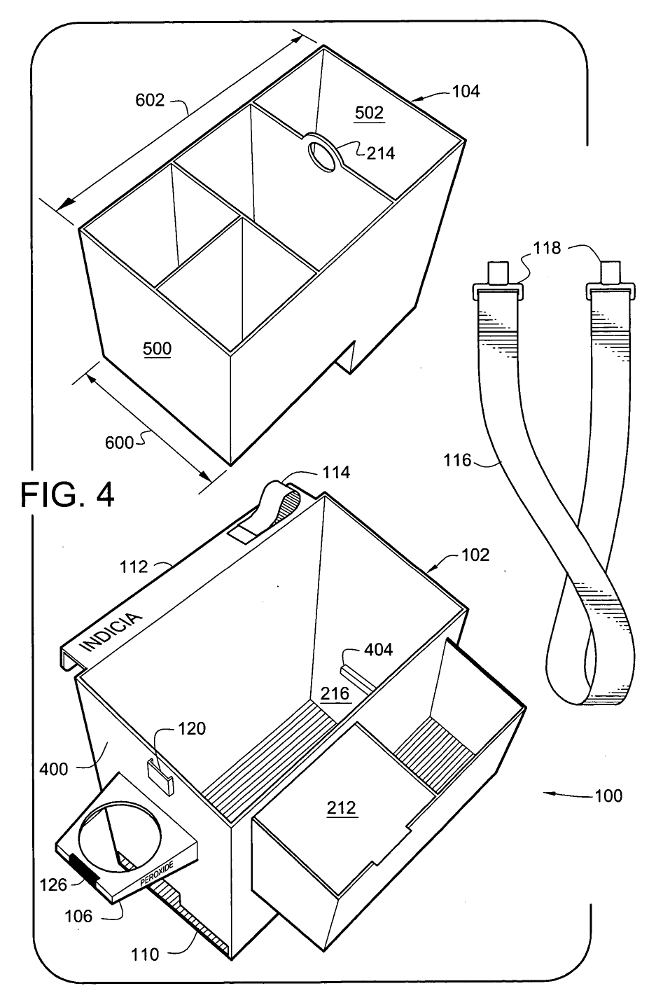

FIG. 1 shows a perspective view of a tracheostomy care container system 100 according to a preferred embodiment of the present invention. Preferably, tracheostomy care container system 100 comprises at least one carrying assistor to help a user carry tracheostomy care container system 100, preferably a strap 116, as shown. Preferably, strap 116 has an adjustable length, preferably so that strap 116 can be used as a shoulder strap and/or handle strap. Preferably, strap 116 is removably attached with attachers 118, which are attached to attacher receptacles 120 (see also FIG. 4). Preferably, attachers 118 operate with a quick release mechanism. Upon reading the teachings of this specification, those with ordinary skill in the art will now understand that, under appropriate circumstances, considering issues such as intended use, market demand, production cost, etc., other carrying assistor arrangements may suffice, such as, for example, omitting strap 116, fixedly attaching strap 116, using other types of strap attachers 118 such as snaps, buckles, clasps, using another type of flexible or rigid handle instead of or in addition to strap, etc.

Preferably, tracheostomy care container system 100 comprises cleaning solution container holder 106 to removably hold cleaning solution container 316, as shown. Preferably, cleaning solution container 316 is a disposable plastic or paper cup for holding cleaning solution (preferably hydrogen peroxide solution). Preferably, cleaning solution container holder 106 holds cleaning solution container 316 securely in place so that cleaning solution container 316 won't tip over or spill. Preferably, cleaning solution container holder 106 comprises cleaning solution identification indicia 126. Preferably, cleaning solution identification indicia 126 comprise colors and/or patterns to easily distinguish cleaning solution container holder 106 from water container holder 108. Preferably, cleaning solution identification indicia 126 comprise the color red. Preferably, cleaning solution identification indicia 126 comprise the word “Peroxide” and/or the word “Solution”. Upon reading the teachings of this specification, those with ordinary skill in the art will now understand that, under appropriate circumstances, considering issues such as intended use, market demand, production cost, etc., other cleaning solution container holder arrangements may suffice, such as, for example, using different colors or words for cleaning solution identification indicia, having cleaning solution container fixed instead of removable, etc.

Preferably, tracheostomy care container system 100 comprises water container holder 108 for removably holding water container 318, as shown. Preferably water container 318 is a disposable plastic or paper cup. Preferably, water container holder 108 holds water container 318 securely in place so that water container 318 won't tip over or spill. Preferably water container holder 108 comprises water identification indicia 128. Preferably, water identification indicia 128 comprise at least one color and/or at least one pattern to easily distinguish water container holder 108 from cleaning container holder 106. Preferably, water identification indicia 128 comprise primarily the color blue. Preferably, water identification indicia 126 comprise the word “Water”. Upon reading the teachings of this specification, those with ordinary skill in the art will now understand that, under appropriate circumstances, considering issues such as intended use, market demand, production cost, etc., other water container holder arrangements may suffice, such as, for example, using different colors or words for water identification indicia, having water container fixed instead of removable, etc.

Preferably tracheostomy care container system 100 comprises rigid material, preferably plastic. Upon reading the teachings of this specification, those with ordinary skill in the art will now understand that, under appropriate circumstances, considering issues such as intended use, market demand, production cost, etc., other materials may suffice, such as, for example, metal, flexible material, fabrics, or combinations of different materials, etc. Preferably, cleanliness of the material used to construct tracheostomy care container system 100 will be easily maintainable.

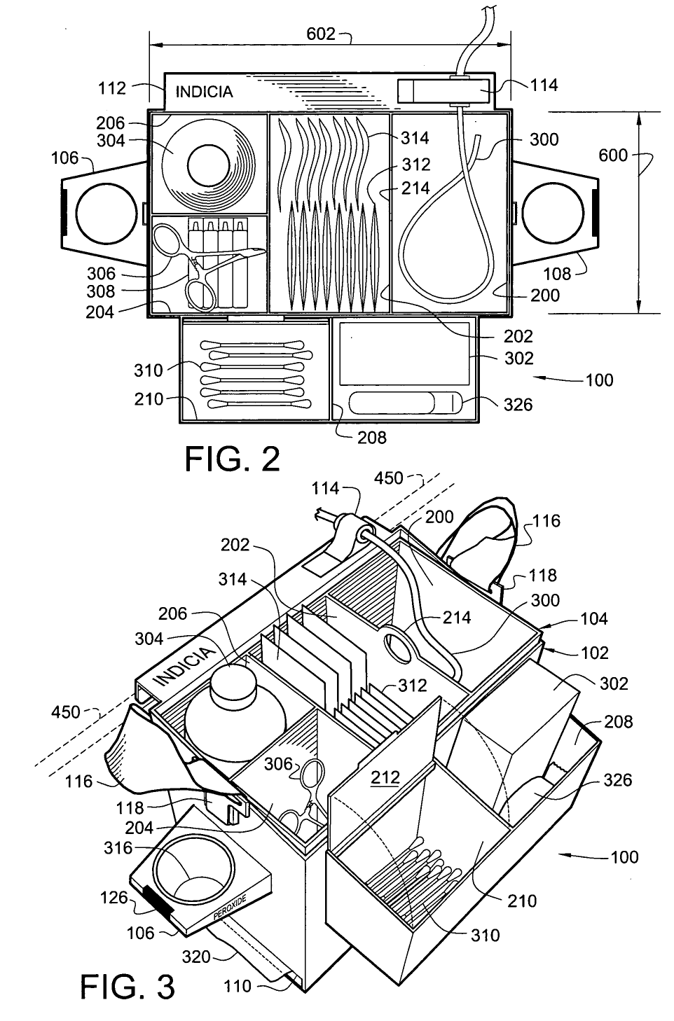

Reference is now made to FIG. 2 and FIG. 3. FIG. 2 shows a top view of tracheostomy care container system 100. FIG. 3 shows a perspective view of tracheostomy care container system 100. Preferably, tracheostomy care container system 100 comprises used catheter container 200. Preferably used catheter container 200 is structured and arranged to store used catheter 300. Preferably used catheter 300 is placed within clean container 322, such as, for example, a clean plastic bag (preferably from within the catheter kit package 320 in which the catheter was initially provided). Preferably catheter 300 and clean container 322 are placed within used catheter container 200. Preferably, used catheter container 200 helps prevent catheter 300 from falling out of clean container 322. Preferably, used catheter container 200 assists in transporting catheter 300 and clean container 322 together with other trach care materials. Preferably, tracheostomy care container system 100 comprises catheter holder 114 to assist in holding and securing used catheter 300 within used catheter container 200, as shown. Preferably, catheter holder 114 comprises hook-and-loop fastening material, such as VELCRO(™), as shown. Upon reading the teachings of this specification, those with ordinary skill in the art will now understand that, under appropriate circumstances, considering issues such as intended use, market demand, production cost, etc., other catheter container arrangements may suffice, such as, for example, keeping catheter container 200 clean so that catheter 300 can be stored in catheter container 200 without clean container 322, using other types of catheter holders such as snaps, elastic, straps, clips, etc.

Preferably, tracheostomy care container system 100 comprises used tracheostomy care item container 202, as shown. Preferably, tracheostomy care item container 202 comprises gauze pad container adapted to contain gauze pads 312 used for tracheostomy care. Preferably, tracheostomy care item container 202 comprises tracheostomy tube holder container adapted to contain tracheostomy tube holders 314 used for tracheostomy care.

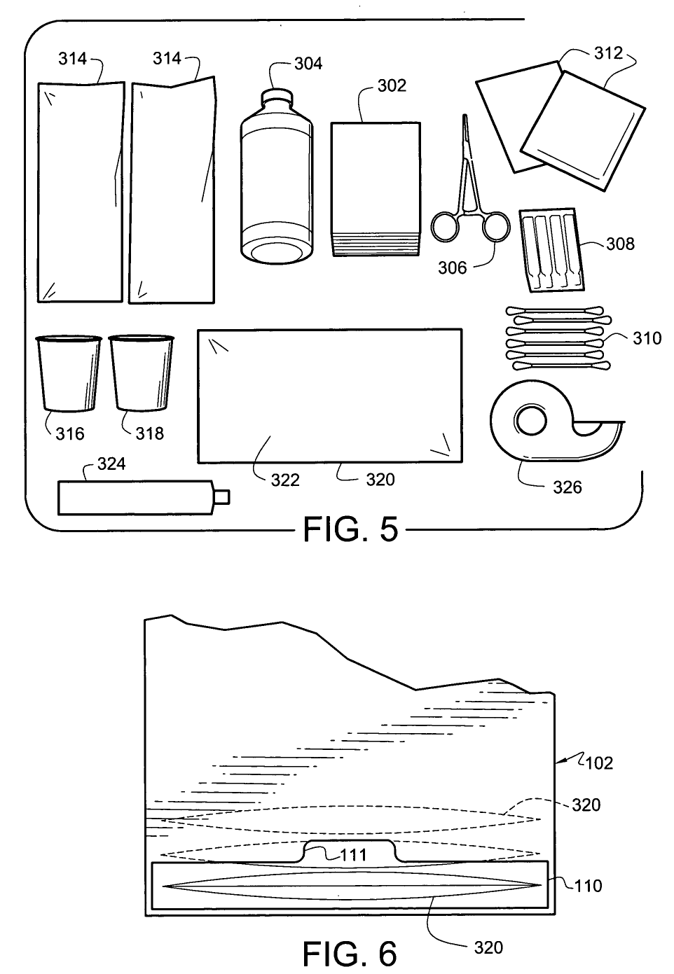

Preferably, tracheostomy care container system 100 comprises hemostat container 204, as shown. Preferably, hemostat container 204 is adapted to contain a hemostat 306. Preferably hemostat container 204 is also adapted to contain saline vials 308 and lubricant 324 (see FIG. 5).

Preferably, tracheostomy care container system 100 comprises hydrogen peroxide container 206, as shown. Preferably, hydrogen peroxide container 206 is adapted to contain a bottle of hydrogen peroxide 304 (which can be diluted to make cleaning solution for tracheostomy care).

Preferably, tracheostomy care container system 100 comprises tracheostomy tube container 208, as shown. Preferably, tracheostomy tube container 208 is adapted to contain a tracheostomy tube 302 (which preferably comprises a package to keep tracheostomy tube 302 sterile). Preferably, tracheostomy tube container 208 is also adapted to contain medical tape 326.

Preferably, tracheostomy care container system 100 comprises cotton swab container 210. Preferably, cotton swab container 210 is adapted to contain a plurality of cotton swabs 310, such as, for example, Q-TIPS(™), as shown. Preferably, cotton swab container 210 comprises lid 212, as shown in FIG. 3.

Upon reading the teachings of this specification, those with ordinary skill in the art will now understand that, under appropriate circumstances, considering issues such as user preference, production cost, etc., other container arrangements may suffice, such as, for example, containers adapted to hold different (or other combinations of) trach care materials, etc.

Preferably, tracheostomy care container system 100 comprises attacher 112 structured and arranged so that tracheostomy care container system 100 can be attached to furniture or other structures, as shown. Preferably, attacher 112 comprises a lip, as shown, which can be used to hang tracheostomy care container system 100 from furniture 450 or other objects, such as, for example, by hooking the lip over the top rail of a crib or bed. Upon reading the teachings of this specification, those with ordinary skill in the art will now understand that, under appropriate circumstances, considering issues such as intended use, market demand, production cost, etc., other attacher arrangements may suffice, such as, for example, using clips, straps, hooks, etc., instead or in addition to a lip, etc.

FIG. 4 shows an exploded perspective view of an empty (without trach care items) tracheostomy care container system 100. Preferably, tracheostomy care container system 100 comprises first holder 102 and second holder 104. Preferably, first holder 102 comprises suction catheter kit container 216 adapted to hold a plurality of suction catheter kits 320 (not shown in FIG. 4, see FIG. 3 and FIG. 6). Preferably, second holder 104 is adapted to nest within first holder 102. Preferably, a plurality of suction catheter kits 320 are stacked within suction catheter kit container 216 and then second holder 104 is nested within first holder interior 402, thereby enclosing suction catheter kits 320 within first holder 102. Preferably, second holder 104 (when nested within first holder 102) rests upon stopper 404 which prevents suction catheter kits 320 from being crushed by second holder 104. Preferably, dispensing slot 110 allows suction catheter kits 320 to be removed from tracheostomy care container system 100 without the need to remove second holder 104 from first holder 102.

Preferably, second holder 104 has a width 600 which is about the same as the width of a standard suction catheter kit 320. Preferably, second holder 104 has a length 602 which is about the same as the length of a standard suction catheter kit 320. Preferably, catheter glove kit container 216 has about the same width 600 and about the same length 602 as second holder 104 (preferably first holder 102 is slightly larger in each dimension to allow nesting, as shown). Preferably, catheter glove kit container 216 has a width of about 5-1/2 inches and a length of about 10 inches. Upon reading the teachings of this specification, those with ordinary skill in the art will now understand that, under appropriate circumstances, considering issues such as intended use, market demand, production cost, etc., other holder arrangements may suffice, such as, for example, alternate lengths and widths, holders which are not nested, holders which are connected in an alternate fashion (such as stacked), etc.

Preferably second holder 104 comprises grip 214 to assist in removing (un-nesting) second holder 104 from first holder 102.

FIG. 5 shows a collection of trach care items which preferably comprise tracheostomy care container system 100. Preferably, tracheostomy care container system 100 comprises tracheostomy tube 302, hydrogen peroxide 304, hemostat 306, saline vials 308, cotton swabs 310, gauze pads 312, tracheostomy tube holder 314, cleaning solution container 316, water container 318, suction catheter kit 320, lubricant 324 (such as, for example, KY jelly) and medical tape 320. Preferably, gauze pads 312 are pre-cut for use in trach care. Preferably, suction catheter kit 320 comprises a suction catheter and glove sealed within clean container 322. Preferably clean container 322 is sterile.

FIG. 6 shows a partial cutaway of tracheostomy care container system 100. Preferably, tracheostomy care container system 100 comprises dispensing slot 110. Preferably, dispensing slot 110 is adapted to allow suction catheter kits 320 to be removed one at a time from suction catheter kit container 216. Preferably, dispensing slot 110 comprises notch 111 to allow a users fingers greater access in grasping one suction catheter kit 320 (while the remaining suction catheter kits 320 remain within suction catheter kit container 216).

Tracheostomy care container system 100 allows trach care and/or catheter suctioning trach care to be performed more conveniently and efficiently by assembling the above mentioned trach care items so they are easily accessible and easily transportable.

The following steps are preferably performed for trach care: Preferably, trach tube holder 314 are cut to the appropriate length. Preferably, cleaning solution (preferably comprising ½ portion hydrogen peroxide 304 and ½ portion sterile water) is poured into cleaning solution container 316. Preferably, sterile water is poured into water container 318. Preferably, the skin around the trach tube is cleaned with cotton swabs 310 soaked in cleaning solution. Preferably, the skin around the trach tube is then rinsed with cotton swabs 310 soaked in sterile water. Preferably, the skin around the trach tube is then dried with gauze 312 or dry cotton swabs 310. Preferably, trach tube holders 314 are then replaced. Preferably gauze 312 is tucked under the trach tube flush with skin.

The following steps are preferably performed for suctioning trach care: Preferably, remove suction catheter kit 320 from tracheostomy care container system 100. Preferably, put on glove from suction catheter kit 320 (for sterility). Preferably, connect suction catheter to machine tubing of suction machine (not shown). Preferably, inject sterile saline from saline vial 308 into the trach tube (if needed to loosen thick or dry mucus which is difficult to suction). Preferably, perform suctioning using catheter from suction catheter kit 320. Preferably, when finished suctioning, suction cleaning solution from cleaning solution container 316 through catheter (until catheter is clean of mucus, etc.). Preferably, place catheter 300 back within suction catheter kit container 216 and place both within used catheter container 200 for later reuse. Secure catheter 300 in place with catheter holder 114 to prevent catheter from slipping/falling out. Upon reading the teachings of this specification, those with ordinary skill in the art will now understand that, under appropriate circumstances, considering issues such as condition of the stoma, complications, special medical conditions, etc., other trach care procedures may suffice, such as, for example, using additional (or fewer) trach care items, etc.

Although applicant has described applicant's preferred embodiments of this invention, it will be understood that the broadest scope of this invention includes such modifications as diverse shapes and sizes and materials. Such scope is limited only by the below claims as read in connection with the above specification.

Further, many other advantages of applicant's invention will be apparent to those skilled in the art from the above descriptions and the below claims.

1) A tracheostomy care container system comprising, in combination:
a) at least one suction catheter kit container adapted to contain a plurality of suction catheter kits; and
b) at least one used suction catheter container adapted to contain a used suction catheter within a substantially clean environment so that such used suction catheter can be used again.

2) The tracheostomy care container system according to claim 1 further comprising at least one suction catheter holder to hold such used suction catheter within said at least one used suction catheter container.

3) The tracheostomy care container system according to claim 2 wherein said at least one suction catheter holder comprises hook and loop fastening material.

4) The tracheostomy care container system according to claim 1 further comprising:
a) at least one tracheostomy care item container adapted to contain at least one tracheostomy care item;
b) wherein said at least one tracheostomy care item is selected from the group consisting essentially of
i) gauze
ii) a cotton swab
iii) a hemostat
iv) a saline vial
v) a tracheostomy tube holder
vi) a tracheostomy tube
vii) medical tape
viii) lubricant jelly.

5) The tracheostomy care container system according to claim 4 further comprising:
a) at least one gauze pad;
b) at least one cotton swab;
c) at least one tracheostomy tube holder;
d) at least one tracheostomy tube;
e) a reservoir for water; and
f) a reservoir for cleaning solution.

6) The tracheostomy care container system according to claim 1 wherein said at least one used suction catheter container is adapted to nest within said at least one suction catheter kit container.

7) The tracheostomy care container system according to claim 2 further comprising at least one carrying assistor selected from the group consisting essentially of:
a) a shoulder strap
b) a handle.

8) The tracheostomy care container system according to claim 1 further comprising at least one attacher structured and arranged so that the tracheostomy care container system can be attached to hang from furniture.

9) The tracheostomy care container system according to claim 8 further comprising:
a) at least one water holder adapted to hold at least one water container; and
b) at least one cleaning solution holder adapted to hold at least one cleaning solution container.

10) The tracheostomy care container system according to claim 9 further comprising:
a) at least one tracheostomy care item container adapted to contain at least one tracheostomy care item;
b) wherein said at least one tracheostomy care item is selected from the group consisting essentially of
i) gauze
ii) a cotton swab
iii) a hemostat
iv) a saline vial
v) a tracheostomy tube holder
vi) an tracheostomy tube
vii) medical tape
viii) lubricant jelly.

11) The tracheostomy care container system according to claim 10 wherein said at least one suction catheter kit container comprises at least one dispensing slot adapted to dispense suction catheter kits.

12) The tracheostomy care container system according to claim 10 wherein said suction catheter kit container comprises:
a) a suction catheter kit container length, wherein said suction catheter kit container length is about 10 inches; and
b) a suction catheter kit container width, wherein said suction catheter kit container width is about 5-1/2 inches.

13) The tracheostomy care container system according to claim 9 further comprising:
a) at least one cotton swab container adapted to contain at least one cotton swab;
b) wherein said at least one cotton swab container comprises a reclosable lid;
c) at least one gauze pad container adapted to contain at least one gauze pad; and
d) at least one hydrogen peroxide container adapted to contain at least one bottle of hydrogen peroxide.

14) The tracheostomy care container system according to claim 13 further comprising:
a) at least one hemostat container adapted to contain at least one hemostat;
b) at least one tracheostomy tube holder container adapted to contain at least one tracheostomy tube holder; and
c) at least one tracheostomy tube container adapted to contain at least one tracheostomy tube.

15) A tracheostomy care container system comprising, in combination:
a) at least one suction catheter kit container adapted to contain a plurality of suction catheter kits;
b) wherein said suction catheter kit container comprises
i) a suction catheter kit container length, wherein said suction catheter kit container length is about as long as a standard suction catheter kit, and
ii) a suction catheter kit container width, wherein said suction catheter kit container width is about as wide as a standard suction catheter kit; and
c) at least one carrying assistor selected from the group consisting essentially of
i) a shoulder strap
ii) a handle.

16) The tracheostomy care container system according to claim 15 wherein said at least one carrying assistor is structured and arranged to be detachable.

17) The tracheostomy care container system according to claim 15 wherein:
a) said suction catheter kit container length is about 10 inches; and
b) said suction catheter kit container width is about 5-1/2 inches.

18) A tracheostomy care container system, which can be attached to furniture, comprising, in combination:
a) at least one suction catheter kit container adapted to contain a plurality of suction catheter kits; and
b) at least one attacher structured and arranged so that the tracheostomy care container system can be attached to hang from the furniture.

19) The tracheostomy care container system according to claim 18 wherein said at least one attacher comprises at least one lip.

20) The tracheostomy care container system according to claim 19 wherein said at least one lip is adapted for attachment to the upper rail of a crib.

21) A tracheostomy care container system comprising, in combination:
a) at least one suction catheter kit container adapted to contain a plurality of suction catheter kits; and
b) at least one used suction catheter container adapted to contain a used suction catheter within a substantially sterile environment so that such used suction catheter can be used again;
c) wherein said at least one suction catheter kit container comprises at least one dispensing slot adapted to dispense suction catheter kits.

22) The tracheostomy care container system according to claim 21 wherein said at least one used suction catheter container is adapted to nest within said at least one suction catheter kit container.

23) A tracheostomy care container system comprising, in combination:
a) at least one suction catheter kit container adapted to contain at least one suction catheter kit;
b) at least one water holder adapted to hold at least one water container; and
c) at least one cleaning solution holder adapted to hold at least one cleaning solution container.

24) The tracheostomy care container system according to claim 23 wherein:
a) said at least one water holder comprises at least one water container; and
b) said at least one cleaning solution holder comprises at least one cleaning solution container.

25) The tracheostomy care container system according to claim 24 wherein:
a) said at least one water container comprises at least one cup; and
b) said at least one cleaning solution container comprises at least one cup.

26) The tracheostomy care container system according to claim 23 wherein:
a) said at least one water holder is adapted to removably hold at least one disposable cup; and
b) said at least one cleaning solution holder is adapted to removably hold at least one disposable cup.

27) The tracheostomy care container system according to claim 23 wherein:
a) said at least one water holder comprises water identification indicia; and
b) said at least one cleaning solution holder comprises cleaning solution identification indicia.

28) The tracheostomy care container system according to claim 27 wherein said water identification indicia comprises a different color than said cleaning solution identification indicia.

29) The tracheostomy care container system according to claim 28 wherein said water identification indicia comprises primarily the color blue.

30) The tracheostomy care container system according to claim 28 wherein said cleaning solution identification indicia comprises primarily the color red.

31) The tracheostomy care container system according to claim 28 wherein said water identification indicia comprises at least one word.
